(19) United States
(12) Patent Application Publication
(10) Pub. No.: US 2006/0073492 A1
(43) Pub. Date: Apr. 6, 2006

(54) Prediction and predisposition of MBL gene to bronchial asthma with allergic rhinitis

(76) Inventors: Puranam Usha Sarma, New Delhi (IN); Taruna Madan, New Delhi (IN); Savneet Kaur, New Delhi (IN)

Correspondence Address: FROMMER LAWRENCE & HAUG, 745 FIFTH AVENUE- 10TH FL., NEW YORK, NY, 10151, US

(21) Appl. No.: 11/145,772

(22) Filed: Jun. 6, 2005

(30) Foreign Application Priority Data
Jun. 4, 2004 (IN) 1038DEL2004

(51) Int. Cl. C12Q 1/68; C07H 21/04; A61K 48/00

(52) U.S. Cl. 435/6; 514/44; 536/24.3

(57) ABSTRACT

The present invention relates to detection of allelic variants of MBL gene useful for prediction of predisposition to bronchial asthma with allergic rhinitis. The invention also provides for detecting allelic variants in MBL gene and their use in diagnosis and prediction of an individual's susceptibility to bronchial asthma with allergic rhinitis. The utility of the invention is in applications such as molecular diagnosis and prediction of an individual's susceptibility to bronchial asthma with allergic rhinitis.

### CROSS REFERENCE TO RELATED APPLICATIONS

This application claims priority to Indian Patent Application No. 1038/DEL/2004, filed on Jun. 4, 2004.

The foregoing application, as well as all documents cited in the foregoing application (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to detection of allelic variants of MBL gene useful for prediction of predisposition to bronchial asthma with allergic rhinitis. The invention also provides for detecting allelic variants in MBL gene and their use in diagnosis and prediction of an individual's susceptibility to bronchial asthma with allergic rhinitis. The utility of the invention is in applications such as molecular diagnosis and prediction of an individual's susceptibility to bronchial asthma with allergic rhinitis.

### BACKGROUND OF THE INVENTION

Asthma, Allergic Rhinitis and Candidate Genes

Asthma is a disease of multiple origins characterized by reversible airway obstruction, eosinophil infiltration into the lung, airway hyper-responsiveness, epithelial damage and mucous secretion. It is a complex genetic disorder with a heterogeneous phenotype, largely attributed to the interactions among many genes and between these genes and the environment. Allergic rhinitis (or commonly called hay fever) is an inflammation or irritation of the mucous membrane that line the nose. Asthma and allergic rhinitis are manifestations of a single syndrome, the chronic allergic respiratory syndrome, occurring in two parts of the respiratory tract, nasal and the lower airways. Once manifested, the two conditions track in parallel in terms of severity [Bousquet et al, 2001]. Allergic rhinitis is an important risk factor for asthma [Guerra et al, 2002, Halpern et al, 2004] that patients who have both allergic rhinitis and asthma suffer more debilitating chest symptoms than patients with asthma alone.

Asthma and allergic rhinitis are strongly familial, and are due to interactions_between genetic and environmental factors. Genes predispose to allergy and asthma because their DNA sequences contain polymorphisms (variants), which alter gene function. Genes causing disease may be identified either by the process known as positional cloning or by examining the candidate genes. Candidate genes are genes, which are already known to be involved in the pathways contributing to the disease. The role of candidate genes may be assessed by defining polymorphisms within the respective genes, and testing for associations with disease. Genes may cause susceptibility to allergy and asthma through a number of different pathways. They may predispose in general to allergy, or they may influence the specific IgE response to individual allergens, or may influence bronchial hyper-responsiveness and enhance inflammation independent of allergy. Various candidate genes have been associated with asthma and allergic rhinitis. For_example, two coding polymorphisms have been identified in exon 6 of the beta chain of the high affinity receptor for IgE (FcεR1β) that influence susceptibility to asthma [Shirakawa et al, 1994]. The 5q23-31 region of the human genome harbors several genes implicated in the pathogenesis of asthma and atopy [Marsh et al, 1994, Amelung et al, 1995, Walley et al, 1997]. These include the genes coding for interleukins 3 (IL-3), IL-4, IL-5, IL-9, IL-12b, IL-13, the glucocorticoid receptor and the β2 adrenergic receptor. An association between HLA class I haplotypes and IgE responses have also been reported [Levine et al, 1972]. Also, an association of HLA DRB1 alleles with allergic rhinitis has been reported (Zhonghua et al 1999). Variations in the levels of the pro inflammatory cytokine, tumor necrosis factor alpha (TNF-α), which are linked to polymorphisms in the TNF gene complex, have also been associated with asthma. [Messer et al, 1991, Gentile et al, 1994]. The study by Laryngoscope et al, 2004 indicated that the -403 and -28 alleles in the RANTES promoter region belong to the predictor gene set for allergic rhinitis.

Since, bronchial asthma is a multifactorial disease, a large number of genes are associated with it. Contrary to single gene disorders where a single gene shows two alleles wherein one is protective and the other is predisposing to disease, the alleles in bronchial asthma and allergic inflammation may not show all or none effect. They are contributing partially to the disease susceptibility or protection.

Recent insights into the complex mechanisms of asthma and rhinitis suggest that genetic variability in innate immunity genes encoding its components may play a role in the development of asthma and related diseases [Lazarus et al, 2002]. Eder et al, 2004 suggest that genetic variation in TLR2 is a major determinant of the susceptibility to asthma and allergies.

The complement system is an important constituent of innate immunity. Complement activation forms the first line defense against pathogenic organisms and contributes to many innate immune system functions including inflammation, opsonization and lysis. It occurs via three distinct mechanisms, the classical pathway, the alternative pathway and the lectin pathway. All three pathways culminate in the production of opsonins, membrane attack complex and anaphylatoxins such as C3a and C5a. A plethora of data has accumulated directing towards a pathogenic role of complement activation products C3a and C5a as potential effectors in Type 1 hypersensitivity reactions, including urticaria, rhinitis and asthma [Kirschfink et al, 1993, Humbles et al, 2000, Drouin et al, 2002]. Studies by Regal et al, 1997 have demonstrated that complement activation makes a significant contribution to systemic inflammation in asthma and rhinitis. Hence genes that are involved in complement activation in humans may contribute towards susceptibility to asthma and allergic rhinitis.

MBL and Disease Associations

Mannan-binding lectin (MBL), an important animal serum lectin structurally similar to the surfactant proteins, is composed of trimeric subunits with a collagenous section and a C-terminal globular carbohydrate-recognizing domain (CRD). MBL recognizes and binds to non-host carbohydrate structures expressed on the surfaces of various pathogens such as yeast, bacteria and viruses via its CRD and the collagenous domain reacts with the C1q receptor (collectin receptor) found on many cells including phagocytes, thus acting as an opsonin [Jack et al, 2001]. MBL initiates the lectin pathway of complement activation, which is independent of C1q and antibody and has been described as a key mechanism for the mammalian acute phase response to infection [Matsushuita et al, 1992]. Its contribution to the host resistance includes opsonization and generation of inflammatory mediators C3a and C5a. These complement products, as mentioned earlier are important mediators of the asthmatic reactions.

The gene for MBL is located at chromosome 10q11.2-q23. It consists of four exons interrupted by three introns. Exon 1 encodes the N-terminal signal peptide, a cysteine rich collagen domain that interacts with the phagocytes and is important for complement activation [Taylor et al, 1989]. Three major mutations [codon 52 (CGT to TGT), codon 54 (GGC to GAC) and codon 57 (GGA to GAA), resulting in Arg to Cys, Gly to Asp and Gly to Glu aminoacid substitutions], found respectively in exon 1 of MBL along with two promoter polymorphisms [-550 (G to C), -221(G to C) and a SNP at +4 of the 5′ untranslated region (C to T) alter the structural assembly of MBL and lead to significantly reduced levels of the functional protein circulating in the serum Madsen et al, 1995]. The polymorphisms in the MBL gene and thus varied levels of complement fixing MBL in the serum have been associated with susceptibility or protection of a wide spectrum of clinical manifestations [Turner et al, 1995, Eisen et al, 2003]. For example, Peterslund et al, 2001, demonstrated that bacteremia and pneumonia are significantly associated with MBL polymorphisms in chemotherapy-treated Danish hematology patients. A strong clinical association between MBL variant alleles and meningococcal disease susceptibility has been shown by Hibberd et al, 1999 in pediatric patients in a United Kingdom-wide study of patients with meningococcal disease. In a study conducted in Oxfordshire, England, by Roy et al, 2002, it has been observed that higher proportions of patients with invasive pneumococcal infection were homozygous for MBL variant alleles. In another study by Hoal-Van Helden et al, 1999, involving a black South African population, the codon 54 polymorphism of MBL gene was observed to be associated with protection from tuberculous meningitis and pulmonary disease. The presence of variant alleles of MBL has also been associated with reduced duration of survival in HIV-infected patients [Garred et al, 1997]. Also, codon 54 and codon 52 polymorphisms of MBL gene are known to be associated with chronic hepatitis B virus (HBV) infection in Japanese and Vietnamese children [Hakozaki et al, 2002, Song le et al, 2003]. Among fungal infections, chronic pulmonary necrotizing aspergillosis, a rare progressive infection caused by Aspergillus fumigatus has been shown to be associated with polymorphisms of the MBL gene by Crosdale et al, 2001. In another study conducted by Luty et al, 1998, on Gabonese children, it has been observed that children with severe malaria had more MBL structural gene mutations as compared with children who had mild malaria.

There are other studies as well, wherein role of varied serum of MBL levels in children with respiratory tract infections and allergies have been shown. In such studies MBL deficiency alters the development of fungal asthma: effects on airway response, inflammation and cytokine (Scand. J Immunol, 2005: 61(5): 466-74). Likewise Nagey et al [J. Allergy Clin Immunol Vol. 113 (3), page 569], show that variant MBL alleles at codon 52, 54 and 57 in asthmatic infected with Chlamydia pneumoniae. On the other hand the present invention relates to a novel polymorphism of human MBL gene at 1011 position on intron of MBL gene and provides specific primers suitable for detecting the polymorphism for applications such as molecular diagnosis and predication of an individual's disease susceptibility to bronchial asthma with allergic rhinitis or otherwise, and/or the genetic analysis of MBL gene in Indian population. The findings of the reported stuffy and the present invention are very different since the SNP at 1011 position of MBL in intron 1, has not been reported earlier including asthma in any reported studies on MBL polymorphisms. The results of the present study are different that what has been reported in earlier studies of Hogaboam et al (2004); J Leukoc Biol. Vol. 75, pages 16-22 and Koh et al (2005) J Asthma Allergy Clin. Immuno. Vol. 25(1); pages 16-22.

The prior art is lacking in any method that associates the polymorphisms of MBL gene to the susceptibility of bronchial asthma and/or allergic rhinitis.

The prior art is also lacking in any study that correlates the substructure of MBL with predisposition to bronchial asthma and/or allergic rhinitis.

To the best of our knowledge, this is the first demonstration that relates to the application of intronic SNP (G1011A) in human MBL gene for use such as molecular diagnosis and prediction of an individual's disease susceptibility to bronchial asthma with allergic rhinitis or otherwise.

The novelty of the present invention is in providing a polymorphism of MBL gene and associating it with high MBL levels, complement activity and for prediction of an individual's predisposition to bronchial asthma with allergic rhinitis.

### OBJECTS OF THE INVENTION

The main object of the present invention is to provide allelic variants of human MBL gene useful for prediction of predisposition to bronchial asthma with allergic rhinitis. Another object is to provide allele specific primers useful for detection of novel polymorphism of human MBL gene.

Another object of the invention is to provide a method for establishing association of novel allelic variants of MBL gene with disease susceptibility.

Yet another object of the invention is to provide a method for screening individuals for predisposition to bronchial asthma with allergic rhinitis.

Still another object of the invention is to establish association of novel polymorphism of MBL gene with high MBL levels and complement activity.

One more object of the present invention provides pharmacogenetic markers for detection of novel allelic variants of human MBL gene.

### SUMMARY OF THE INVENTION

The present invention relates to novel polymorphism of human MBL gene and provides specific primers suitable for detecting the polymorphism for applications such as molecular diagnosis and prediction of an individual's disease susceptibility to bronchial asthma with allergic rhinitis or otherwise, and/or the genetic analysis of MBL gene in Indian population. The invention also relates to the association of MBL gene variants in the patients with high MBL levels and activity in the plasma. Asthma, a chronic inflammatory disorder of the airways, represents a complex interplay of genetic susceptibility and environmental exposures. Recent evidence indicates that, in addition to IgE and T_{H}2 mediated events; the complement system is also involved in modulating susceptibility to asthma. Complement derived inflammatory mediators; C3a and C5a anaphylatoxins are able to mimic the hallmarks of bronchial asthma, which includes early-phase bronchoconstriction with subsequent eosinophil infiltration. The interaction of these complement fragments with their corresponding receptors on basophils provide critical immunomodulatory signals to the adaptive immune system, resulting in the production of IL-4, IL-13 and the cysteinyl leukotrienes, leading to potent bronchoconstriction and development of allergic inflammation. Allergens activate all the three complement pathways, classical, alternative and lectin pathway. The role of classical and alternative pathways in bronchial asthma has been assessed. However, the contribution of the lectin pathway of complement initiated by a multifunctional C-type lectin, mannan-binding lectin (MBL) in asthma and allergic inflammation has not been addressed so far. MBL is also known to bind various pathogenic organisms and allergens and modulate the levels of proinflammatory cytokines and hence it may have a role in the development of asthma and allergic airway inflammation.

MBL is synthesized in the liver and secreted in the blood stream. Individual humans differ 1000-fold in plasma MBL concentration. Six biallelic SNPs in the MBL promoter and exon 1, have been proposed to contribute to the variations in MBL levels. These polymorphisms alter the structural assembly of MBL and lead to significantly reduced levels of the protein in plasma. Varied amounts of MBL have been associated with predisposition to various bacterial and fungal infections, autoimmune disorders and inflammatory diseases. In view of the polymorphisms in the collagen region of MBL leading to the varied levels of the functional protein in the plasma and affecting a number of clinical manifestations, polymorphisms were screened in exon 1 and part of intron 1 of MBL in patients of bronchial asthma with allergic rhinitis and unrelated age-matched controls. An intronic polymorphism at nucleotide position 1011 (G/A, in intron 1) was observed to be significantly associated with patients of bronchial asthma with allergic rhinitis. Patients with A allele at position 1011 showed decreased lung performance (percent FEV1) and increased MBL levels, activity and peripheral blood eosinophilia. The novelty of the present invention is in associating novel polymorphism of MBL gene with high MBL levels, complement activity, and for prediction of an individual's predisposition to bronchial asthma with allergic rhinitis.

The Table 1 provides the nucleotide positions pf MBL gene sequence as in GenBank AF080508. The FIG. 1 shows the schematic representation of the SNP in MBL gene. The arrow line shows the relative location of the polymorphic site in intron of the MBL gene.

TABLE I

| | | | | |
|---|---|---|---|---|
| Primers | Location | S/AS | Location | Sequence |

| | | | | |
|---|---|---|---|---|
| SEQ ID NO:1 | 5′UTR | S | 618-636 | 5′ TGCACCCAGATTGTA |
| (MBL F) | | | | TAGGAC 3′ |
| SEQ ID NO:2 | Intron 1 | AS | 1016-1034 | 5′ AGCTGAATCTCTG |
| (MBL R) | | | | TTTTGA 3′ |

S: Sense, AS: Antisense, Nt.: Nucleotide

### DETAILED DESCRIPTION

The present invention relates to the detection of allelic variants of the human MBL gene and their utility in predicting an individual's susceptibility to bronchial asthma with allergic rhinitis.

In the present invention a stretch of 417 bp from position 618-1034 of MBL (Gene position as per AF08058; FIG. 1), comprising whole of exon 1 (256 bp) and 133 bp out of 662 bp of intron was PCR amplified using the forward primer MBL F (SEQ. ID No.1) and reverse primer MBL R (SEQ. ID No. 2). Further, allelic variants having polymorphisms of G/A at 1011 position in the intron 1 of human MBL gene useful for prediction of predisposition to bronchial asthma with allergic rhinitis were identified. There allelic variants G/A having SEQ ID No. 3 and 4, of human MBL gene useful for prediction of predisposition to bronchial asthma with allergic rhinitis. Further, the allele G in SEQ ID No. 3 is associated with low risk and allele A of SEQ ID No. 4 is associated with high risk (OR=5.8696, p=0.0024) to bronchial asthma with allergic rhinitis. The invention further provides association of A allele of novel polymorphism G/A at 1011 position of MBL gene, with high MBL levels (p<0.001) and complement activity (p=0.006).

Polymorphism of the Invention:

As a first step to the present invention, the applicants carried out the PCR amplification of exon 1 and part of intron 1 of human MBL gene (GenBank Acc. No. AF080508) having SEQ ID NO: 3.

The sequence of the PCR amplified product is:

5′tgcacccagattgtaggacagagggcatgctcggtaaatatgtgttca
ttaactgagattaaccttccctgagttttctcacaccaaggtgaggacca
tgtccctgtttccatcactccctctccttctcctgagtatggtggcagcg
tcttactcagaaactgtgacctgtgaggatgcccaaaagacctgccctgc
agtgattgcctgtagctctccaggcatcaacggcttcccaggcaaagatg
ggcgtgatggcaccaagggagaaaagggggaaccaggtacgtgttgggct
gttctgtctctgcaattctttaccttccagaggaaactgcctggggatat
gaggagactgatgtcctatttgagtatatttttctcaactatactgtaac
tcaaaacagagattcagct 3′

The oligonucleotide primers are shown in bold.

The present invention provides a sequence for the allelic variant of human MBL gene comprising of the following SNP compared with the human MBL gene sequence in the database.

TABLE II

| Site of change | Base change |
|---|---|
| Nucleotide position 1011 | G-A |

The site of change is in accordance with the human MBL gene sequence in the database (GenBank Acc No. AF080508).

The nucleotide sequence in SEQ ID No.4 is the allelic variant of human MBL gene having the SNP G/A in intron 1 as listed in table-II is of SEQ ID NO: 4:

5′tgcacccagattgtaggacagagggcatgctcggtaaatatgtgttca
ttaactgagattaaccttccctgagttttctcacaccaaggtgaggacca
tgtccctgtttccatcactccctctccttctcctgagtatggtggcagcg
tcttactcagaaactgtgacctgtgaggatgcccaaaagacctgccctgc
agtgattgcctgtagctctccaggcatcaacggcttcccaggcaaagatg
ggcgtgatggcaccaagggagaaaagggggaaccaggtacgtgttgggct
gttctgtctctgcaattctttaccttccagaggaaactgcctggggatat
gaggagactgatgtcctatttgagtatatttttctcaactatact a*ta
actcaaaacagagattcagct 3′

The above sequence is the amplified gene product, wherein the intronic SNP G/A at nucleotide position 1011 is marked with an asterisk and the oligonucleotide primers are shown in bold.

Association of the Novel SNP with the Disease.

Analysis of SNP in 40 normal and 38 bronchial asthma with allergic rhinitis patient chromosomes revealed that two alleles, possible with the SNP in a biallelic polymorphic system, were observed. The frequency in normal and bronchial asthma with allergic rhinitis patient chromosomes is summarized in Table-III.

TABLE III

| | | SNP at 1011 position | |
|---|---|---|---|
| | No. of chromosomes (N) studied | % G allele | % A allele |
| Normal | 40 | 90 | 10 |
| Patient | 38 | 60.5 | 39.4 |

TABLE IV

| | SNP (G vs. A) at 1011 position |
|---|---|
| ODDS RATIO (patient vs. normal) | 5.8696 |
| Chi-square | 9.188 |
| p-value | 0.0024 |

Further, studies on bronchial asthma with allergic rhinitis patient chromosomes revealed a significant difference in the distribution of the SNP in the chromosomes of normal and the patients of bronchial asthma with allergic rhinitis (Table-IV).

The peripheral blood eosinophilia, involved in onset and maintenance of bronchial asthma, was significantly higher in patients with A allele genotype at 1011 position than those with G allele at 1011 position. The percent-predicted FEV₁, a measure of lung performance, in patients with A allele at 1011 position was lower than patients with G allele at 1011 position. (Table-V).

TABLE V

| | Patients with | Patients with | Patients with | |
|---|---|---|---|---|
| | G/G at 1011, | G/A at 1011, | A/A at 1011, | |
| | n = 7 Mean | n = 9 Mean | n = 3 Mean | |
| | (S.D.) (a) | (S.D.) (b) | (S.D) (c) | p Value |
| FEV₁ (%) | 74.5 (16.2) | 68.5 (17.5) | 45.6 (29.2) | p_{ac} = |
| | | | | 0.073 |
| Eosino- | 5.4 (3.359) | 8 (3.122) | 15.3 (4.163) | p_{ac} = |
| philia (%) | | | | 0.004 |

S.D., Standard deviation,
n = number of patients

Association of the Novel SNP with MBL Levels and Complement Activity

High MBL levels and complement activity were present in patients with A allele at 1011 position of MBL gene, and low MBL levels and complement activity were present in patients with G allele. (FIG. 2).

Accordingly, the main embodiment of the present invention relates to a allelic variants of human MBL gene having SEQ ID NO. 4.

Another embodiment of the present invention relates to allelic variants, wherein said variants are associated with bronchial asthma with allergic rhinitis.

Yet another embodiment of the present invention relates to allelic variants wherein the allele A at 1011 position of MBL gene is associated with high MBL levels and complement activity and allele G is associated with low MBL levels and complement activity.

Still another embodiment of the present invention relates to a method for detection of the novel allelic variants human MBL gene associated with bronchial asthma with allergic rhinitis, said method comprising the steps of:
(1) designing and synthesizing specific oligonucleotide primers of SEQ ID Nos. 1 and 2 for PCR amplification of exon 1 and part of intron 1 of human MBL,
(2) amplifying genomic DNA of patients of bronchial asthma with allergic rhinitis and normal control individuals using the above said primers,
(3) sequencing the amplified PCR product and identifying sequence variation computationally,
(4) screening normal control individuals and patients of bronchial asthma with allergic rhinitis for allelic variants by sequencing amplified exon 1 and part of intron 1 of MBL gene,
(5) computing the frequencies of G/A alleles (SNP at position 1011) in normal control individuals and patients of bronchial asthma with allergic rhinitis, and
(6) establishing the association of G/A alleles statistically with the disease, based on the distribution of their allelic frequencies in normal control individuals and patients.

Another embodiment of the present invention relates to the length of said oligonucleotide primers in ranges from 5 to 100 nucleotides.

One more embodiment of the present invention relates to length of oligonucleotide primers wherein the length of the olgionucleotide primers is 19 nucleotides.

Still another embodiment of the present invention relates to a diagnostic kit for the detection of allelic variants of human MBL gene to establish an individual's susceptibility to bronchial asthma with allergic rhinitis, said kit comprising of:
(a) primers of SEQ ID NOs: 1 and 2,
(b) SEQ ID NOs: 3 and 4
(c) optionally along with buffers, reagents and PCT reagents.

One more embodiment of the present invention relates to pharmacogenetic markers for the detection of allelic variants of human MBL gene to establish an individual's susceptibility to bronchial asthma with allergic rhinitis, said markers comprising of primers of SEQ ID NOs: 3 and 4.

### BRIEF DESCRIPTION OF THE DRAWINGS

The manner in which the above- mentioned features, advantages and objects of the invention as well as others which will become clear are attained and can be understood in details by the particular description of the invention are illustrated in the appended drawings, however, that the appended drawings illustrate preferred embodiments of the invention and thereof not be considered limiting in their scope.

In the drawing (s), accompanying the specification:

FIG. 1 is a schematic representation of the SNP in MBL gene. The arrow line shows the relative location of the polymorphic site in Intron 1 of the MBL gene.

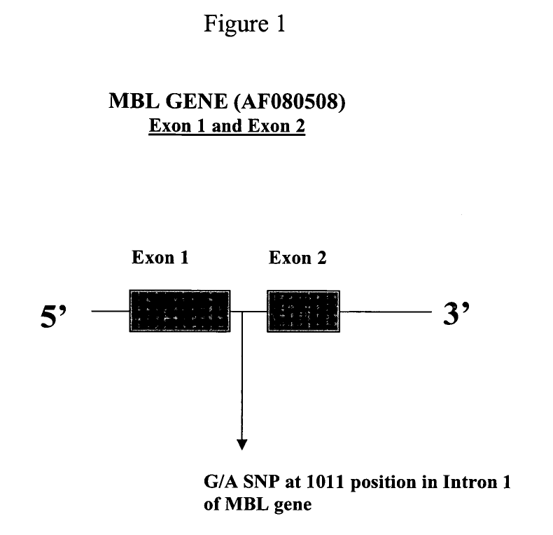

FIGS. 2A and 2B are dot plot representations showing the association of A allele at position 1011 of human MBL gene with high (2A) MBL levels and (2B) complement activity in the plasma of patients having bronchial asthma with allergic rhinitis, having either G or A alleles at position 1011 of MBL gene. Mean values are indicated by lines.

Other and further aspects, features, and advantages of the present invention will be apparent from the following description of the preferred embodiments of the invention given for the purpose of disclosure. Alternative embodiments of the invention can be envisaged by those skilled in the art. All such alternative embodiments are intended to lie within the scope of this invention.

The following examples are given by the way of illustration of the present invention and should not be construed to limit the scope of the present invention.

### EXAMPLES

### Example 1

Identification of Novel Polymorphism of MBL Gene

This example describes the identification of novel polymorphism of human mannan-binding lectin gene by PCR and sequencing using certain oligonucleotide primers. According to the invention DNA was extracted from human peripheral blood leukocytes using a modification of salting out procedure. The concentration of the DNA was determined by measuring the optical density of the sample, at a wavelength of 260 nm. The DNA was then amplified by PCR using the oligonucleotide primers.

(Forward primer, SEQ ID NO: 1)

5′ TGCACCCAGATTGTAGGAC 3′

(Reverse primer, SEQ ID NO: 2)

5′ AGCTGAATCTCTGTTTTGA 3′

Approximately 50 ng of genomic DNA (samples) was amplified in a 50 μl reaction volume containing a final concentration of 5 mM Tris, 25 mM KCl, 0.75 mM magnesium chloride (MgCl2), 0.05% gelatin, 20 pM of each primer and 1.5 U of Taq DNA polymerase. Samples were denatured at 94° C. for 2 min followed by 25 cycles of denaturation (94° C., for 1 min), annealing (52° C., 1 min), extension (72° C., 1 min) and a final extension of 7 min at 72° C. in a Perkin Elmer Gene Amp PCR System 9600. The PCR product was purified by polyethylene glycol and ethanol precipitation and was directly sequenced using dye terminator chemistry on an ABI Prism 377 automated DNA sequencer with the PCR primers.

The PCR product was purified by polyethylene glycol and ethanol precipitation and directly sequenced using gel terminator chemistry on an ABI Prism 377 automated DNA sequencer with PCR prisms. The PCR products were shown to be identical to the human MBL gene sequence in the database submitted by Sastry et al, (acc. no. AF080508), except for the previously mentioned single base change listed in table II.

### Example 2

Nucleotide sequence of the allelic variant of MBL gene.

The nucleotide seq. having SEQ ID No. 4 is the allelic variant of MBL gene derived using the method as described in example 1.

In the above sequence the SNP as given in table II is at nucleotide position 1011.

### Example 3

Patients with A allele at 1011 position are at high risk for bronchial asthma with allergic rhinitis.

A method as described in example 1 is applied to a series of DNA samples extracted from patients of bronchial asthma with allergic rhinitis and normal controls. There is observed a statistically significant difference at position 1011 (p=0.0024) in the frequency distributions of the G and A alleles in normal and bronchial asthma with allergic rhinitis patient MBL chromosomes. The results obtained are summarized in table below.

TABLE IV

| | SNP (G vs. A) at 1011 position |
|---|---|
| ODDS RATIO (patient vs. normal) | 5.8696 |
| Chi-square | 9.188 |
| p-value | 0.0024 |

A strong association of A allele at 1011 position with the disease chromosome indicated that MBL alleles with the A allele at 1011 position are predisposed to the disease. Therefore, this novel SNP in the human MBL gene could be used as a method of establishing individual risk to bronchial asthma with allergic rhinitis. The association of A allele at 1011 position with bronchial asthma with allergic rhinitis was studied in Indian population. However, A allele at 1011 position being at low risk and G allele at 1011 position being at high risk for the disease can be expected to hold true for other human populations also.

### Example 4

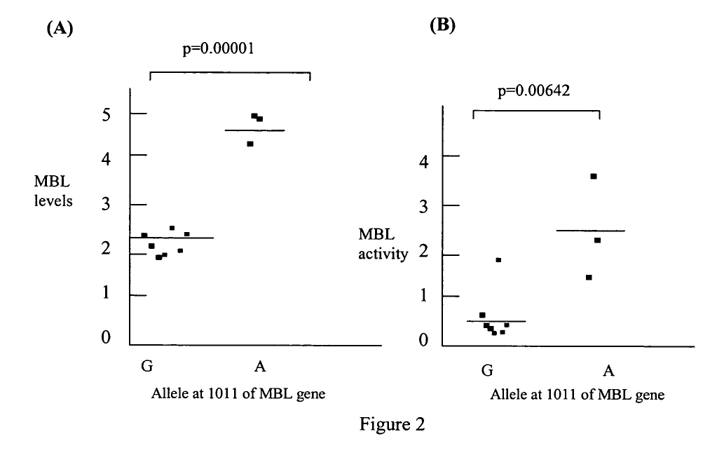

Patients with A allele at 1011 position of MBL gene have high MBL levels (p<0.001) and complement activity (p=0.006) than patients with G allele (FIG. 2).

### Example 5

Diagnostic Kit

The invention further provides diagnostic kit, comprising primers suitable for amplification of MBL gene region containing the polymorphic site are provided, said primers SEQ ID No.1, SEQ ID No.2 wherein primer of SEQ ID No. 1 is forward primer while primer of SEQ ID No. 2 is reverse primer, & compliments thereof or any other pair of suitable primers. Along with the PCR buffers and reagents as given in the Example 1

### ADVANTAGES

The invention shall be useful to establish genotype or base variation of MBL gene. The information may be useful for molecular diagnosis, prediction of an individual's disease susceptibility to bronchial asthma with allergic rhinitis, prognosis and/or the genetic analysis of bronchial asthma with allergic rhinitis gene in a population. The frequency of these variants can also be used to predict the prevalence of disease among various populations.

### REFERENCES

1. Allergic rhinitis and its impact on asthma. J Allergy Clin Immunol. 2001 November;108(5 Suppl):S147-334 Bousquet J, Van Cauwenberge P, Khaltaev N; Aria Workshop Group; World Health Organization.
2. Rhinitis as an independent risk factor for adult-onset asthma. J Allergy Clin Immunol. 2002 March; 109(3):419-25. Guerra S, Sherrill D L, Martinez F D, Barbee R A.
3. Association between atopy and variants of the beta subunit of the high-affinity immunoglobulin E receptor. Nat Genet. 1994 June; 7(2):125-9. Shirakawa T, Li A, Dubowitz M, Dekker J W, Shaw A E, Faux J A, Ra C, Cookson W O, Hopkin J M.
4. Evidence for linkage of total serum IgE and bronchial hyperresponsiveness to chromosome 5q: a major regulatory locus important in asthma. Clin Exp Allergy. 1995 November; 25 Suppl 2:84-8; discussion 95-6 : Bleecker E R, Amelung P J, Levitt R C, Postma D S, Meyers D A
5. Ragweed hay fever: genetic control and linkage to HL-A haplotypes. Science. 1972 Dec. 15;178(66):1201-3. : Levine B B, Stember R H, Fotino M.
6. Polymorphic structure of the tumor necrosis factor (TNF) locus: an NcoI polymorphism in the first intron of the human TNF-beta gene correlates with a variant amino acid in position 26 and a reduced level of TNF-beta production. J Exp Med. 1991 Jan. 1;173(1):209-19. Messer G, Spengler U, Jung M C, Honold G, Blomer K, Pape G R, Riethmuller G, Weiss E H.
7. Association between TNF-alpha and TGF-beta genotypes in infants and parental history of allergic rhinitis and asthma. Hum Immunol. 2004 April;65(4):347-51. Gentile D A, Doyle W J, Zeevi A, Howe-Adams J, Trecki J, Skoner D P.
8. Single nucleotide polymorphisms in innate immunity genes: abundant variation and potential role in complex human disease. Immunol Rev. 2002 December;190:9-25. Review. Lazarus R, Vercelli D, Palmer L J, Klimecki W J, Silverman E K, Richter B, Riva A, Ramoni M, Martinez F D, Weiss S T, Kwiatkowski D J.
9. Toll-like receptor 2 as a major gene for asthma in children of European farmers. J Allergy Clin Immunol. 2004 March;113(3):482-8. Eder W, Klimecki W, Yu L, von Mutius E, Riedler J, Braun-Fahrlander C, Nowak D, Martinez F D; ALEX Study Team.
10. Chemokine RANTES promoter polymorphisms in allergic rhinitis. Kim J J, Lee J H, Jang C H, Kim Y S, Chae S C, Chung H T, Choi T W, Lee J H Laryngoscope. 2004 April;114(4):666-9.
11. Analysis of HLA-DRB1 allele polymorphism for patients with allergic rhinitis. Yang L, Zhang Q, Zhang P. Zhonghua Er Bi Yan Hou Ke Za Zhi. 1999 June;34(3):147-9.
12. Association of low levels of mannose-binding protein with a common defect of opsonization. Lancet II: 1236-1239, 1989. Super, M.; Thiel, S.; Lu, J.; Levinsky, R. J.; Turner, M. W.
13. Interplay between promoter and structural gene variants control basal serum level of mannan-binding protein. J Immunol. 1995 Sep.15; 155(6):3013-20. Madsen H O, Garred P, Thiel S, Kurtzhals J A, Lamm L U, Ryder L P, Svejgaard A
14. Neth O, Jack D L, Dodds A W, Holzel H, Klein N J, Turner M W. Mannose-binding lectin binds to a range of clinically relevant microorganisms and promotes complement deposition. Infect Immun 2000 February;68(2):688-93.
15. Structure and evolutionary origin of the gene encoding a human serum mannose-binding protein. Biochem J. 1989 Sep. 15;262(3):763-71. Taylor M E, Brickell P M, Craig R K, Summerfield J A.
16. Activation of the classical complement pathway by mannose-binding protein in association with a novel C1s-like serine protease. J Exp Med 1992; 176: 1497-502. Matsushita M, Fujita T
17. Mannose-binding lectin: targeting the microbial world. for complement attack and opsonophagocytosis. Immunol Rev 2001; 180: 86-99. Jack D L, Klein N J, Turner M W.
18. Turner M W, Hamvas R M. Mannose-binding lectin: structure, function, genetics and disease associations. J Immunol 1995; 155: 3013-20.
19. Association between deficiency of mannose-binding lectin and severe infections after chemotherapy. Lancet. 2001 Aug. 25;358(9282):637-8. Peterslund N A, Koch C, Jensenius J C, Thiel S.
20. Susceptibility to HIV infection and progression of AIDs in relation to variant alleles of mannose-binding lectin. Lancet 349: 236-240, 1997. Garred, P.; Madsen, H. O.; Balslev, U.; Hofmann, B.; Pedersen, C.; Gerstoft, J.; Svejgaard, A.
21. Association of variants of the gene for mannose-binding lectin with susceptibility to meningococcal disease. Lancet 353: 1049-1053, 1999. Hibberd, M. L.; Sumiya, M.; Summerfield, J. A.; Booy, R.; Levin, M.
22. Mannose-binding lectin gene polymorphisms and hepatitis B virus infection in Vietnamese patients. Mutat Res. 2003 Jan. 28;522(1-2):119-25. Song le H, Binh V Q, Duy D N, Juliger S, Bock T C, Luty A J, Kremsner P G, Kun J F.
23. Mannose-binding lectin gene polymorphisms as a susceptibility factor for chronic necrotizing pulmonary aspergillosis. J Infect Dis. 2001 Sep. 1;184(5):653-6. Epub 2001 Jul. 24. Crosdale D J, Poulton K V, Ollier W E, Thomson W, Denning D W.
24. Mannose-binding protein B allele confers protection against tuberculous meningitis. Pediatr Res. 1999 April;45(4 Pt 1):459-64. Hoal-Van Helden E G, Epstein J, Victor T C, Hon D, Lewis L A, Beyers N, Zurakowski D, Ezekowitz A B, Van Helden P D.
25. A role for the C3a anaphylatoxin receptor in the effector phase of asthma. Nature. 2000 Aug. 31;406(6799):998-1001. Humbles A A, Lu B, Nilsson C A, Lilly C, Israel E, Fujiwara Y, Gerard N P, Gerard C.
26. Absence of the complement anaphylatoxin C3a receptor suppresses Th2 effector functions in a murine model of pulmonary allergy. J Immunol. 2002 Nov. 15;169(10):5926-33. Drouin S M, Corry D B, Hollman T J, Kildsgaard J, Wetsel R A.
27. Role of the complement system in pulmonary disorders. Immunopharmacology. 1997 December;38(1-2):17-25. Review. Regal J F
28. Complement activation and C3 allotype distribution in patients with bronchial asthma. Int Arch Allergy Immunol 1993; 100: 151-5. Kirschfink M, Castro F F, Rother U, Nakhosteen J A, Deppisch R, Schmitz-Schumann M.
29. Impact of mannose-binding lectin on susceptibility to infectious diseases. Clin Infect Dis. 2003 Dec. 1;37(11):1496-505. Epub 2003 Nov. 06. Review. Eisen D P, Minchinton R M.
30. Koh et al (2005). J. Asthma Allergy Clin Immunol. Vol. 25(1), pages 16-22
31. Hogabham et al (2004). J. Leukoc Viol. Vol. 75, pages 805-814
32. Scandinavian J Immunol. (2005) Vol. 61 (5), page 466
33. J. Allergy Clin. Immunol. (2003) Vol. 112, pages 729-734

We claim:

1. Allelic variants of human MBL gene having SEQ ID NO. 4.

2. Allelic variants as claimed in claim 1, wherein said variant is associated with bronchial asthma with allergic rhinitis.

3. Allelic variants as claimed in claim 1, wherein the allele A at 1011 position of MBL gene is associated with high MBL levels and complement activity and allele G is associated with low MBL levels and complement activity.

4. A method for detection of the novel allelic variants human MBL gene associated with bronchial asthma with allergic rhinitis, said method comprising the steps of:
(a) designing and synthesizing specific oligonucleotide primers of SEQ ID Nos. 1 and 2 for PCR amplification of exon 1 and part of intron 1 of human MBL,
(b) amplifying genomic DNA of patients of bronchial asthma with allergic rhinitis and normal control individuals using the above said primers,
(c) sequencing the amplified PCR product and identifying sequence variation computationally,
(d) screening normal control individuals and patients of bronchial asthma with rhinitis for allelic variants by sequencing amplified exon 1 and part of intron 1 of MBL gene,
(e) computing the frequencies of G/A alleles (SNP at position 1011) in normal control individuals and patients of bronchial asthma with allergic rhinitis, and
(f) establishing the association of G/A alleles statistically with the disease, based on the distribution of their allelic frequencies in normal control individuals and patients.

5. A method as claimed in claim 4, wherein the length of said oligonucleotide primers ranges from 5 to 100 nucleotides.

6. A method as claimed in claim 5, wherein the length of said oligonucleotide primers is 19 nucleotides.

7. A diagnostic kit for the detection of allelic variants of human MBL gene to establish an individual's susceptibility to bronchial asthma with allergic rhinitis, said kit comprising of:
(a) primers of SEQ ID NOs: 1 and 2,
(b) SEQ ID NOs: 3 and 4
(c) optionally along with buffers, reagents and PCT reagents.

8. Pharmacogentic markers for the detection of allelic variants of human MBL gene to establish an individual's susceptibility to bronchial asthma with allergic rhinitis, said markers comprising of primers SEQ ID NOs: 3 and 4.


SEQUENCE LISTING (4 sequences)

SEQ ID NO: 1 - DNA, 19 bp, Artificial
  Oligonucelotide primer (1)..(19): Forward Primer
tgcacccagattgtaggac

SEQ ID NO: 2 - DNA, 19 bp, Artificial
  Oligonucelotide primer (1)..(19): Reverse Primer
agctgaatctctgttttga

SEQ ID NO: 3 - DNA, 417 bp, Homo sapiens
  DNA (1)..(417)
tgcacccagattgtaggacagagggcatgctcggtaaatatgtgttcattaactgagattaaccttccctgagttttctcacaccaaggtgaggaccatgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccaggtacgtgttgggctgttctgtctctgcaattctttaccttccagaggaaactgcctggggatatgaggagactgatgtcctatttgagtatatttttctcaactatactgtaactcaaaacagagattcagct

SEQ ID NO: 4 - DNA, 417 bp, Homo sapiens
  DNA (1)..(417): Alleliv variant Nucelotide sequence of MBL gene
tgcacccagattgtaggacagagggcatgctcggtaaatatgtgttcattaactgagattaaccttccctgagttttctcacaccaaggtgaggaccatgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccaggtacgtgttgggctgttctgtctctgcaattctttaccttccagaggaaactgcctggggatatgaggagactgatgtcctatttgagtatatttttctcaactatactataactcaaaacagagattcagct